(19) United States
(12) Patent Application Publication
(10) Pub. No.: US 2025/0134455 A1
(43) Pub. Date: May 1, 2025

(54) APPARATUS AND METHOD FOR EVALUATING XR-BASED UPPER LIMB MOTION CONTROL AND MOTION CONTROL ABILITY

(71) Applicant: KOREA INSTITUTE OF SCIENCE AND TECHNOLOGY, Seoul (KR)

(72) Inventors: Song Joo LEE, Seoul (KR); Hyung Min KIM, Seoul (KR); Kyung-Mi PARK, Seoul (KR); Keun Tae KIM, Seoul (KR); Duguma Teshome Gemechu, Seoul (KR); Ji-Yoon LEE, Seoul (KR); Olga Valerevna KIM, Seoul (KR)

(73) Assignee: KOREA INSTITUTE OF SCIENCE AND TECHNOLOGY, Seoul (KR)

(21) Appl. No.: 18/396,120

(22) Filed: Dec. 26, 2023

(30) Foreign Application Priority Data
Nov. 1, 2023 (KR) 10-2023-0149191

(51) Int. Cl. A61B 5/00; A63B 23/12; G02B 27/01; G06F 3/01; G06T 19/00

(52) U.S. Cl. CPC A61B 5/486; A61B 5/4519; A63B 23/12; G02B 27/017; G06F 3/016; G06T 19/006; G02B 2027/0138

(57) ABSTRACT

The present application relates to an apparatus and method for evaluating XR-based upper limb motion control and motion control ability, the apparatus including a head mount display (HMD) configured to be worn on a user's head part and including a display for providing visual feedback information capable of monitoring a visual cue and a repetitive motion of muscle synergy or a direction and magnitude of a force that are contents for XR (eXtended Reality)-based multi-joint rehabilitation training of an upper limb, a measurement part, a synergy evaluation part, a visual cue control part and a composite biofeedback part.

### CROSS REFERENCE TO RELATED APPLICATION

This application claims priority to and the benefit of Korean Patent Application No. 10-2023-0149191 filed with the Korean Intellectual Property Office on Nov. 1, 2023, the entire contents of which are incorporated herein by reference.

### BACKGROUND OF THE INVENTION

### Field of the Invention

The present application relates to an apparatus and method for evaluating XR-based upper limb motion control and motion control ability, and more particularly, to an apparatus and evaluation for providing composite biofeedback or visual cues in AR or VR, evaluating synergy of muscles by using a force applied by a user by moving upper limbs (hands, arms, and shoulders), identifying weakness, and assisting in rehabilitation training.

### DESCRIPTION OF NATIONAL SUPPORT RESEARCH AND DEVELOPMENT

This research was conducted under the supervision of the Korea Advanced Institute of Science and Technology and funded by the Ministry of Science and ICT through the SW Computing Industry Source Technology Development Project under the SW Computing Industry Source Technology Development Project (Project Name: (BCI-General Directorate/1st Division) Non-invasive BCI Integrated Brain Cognitive Computing SW Platform Technology Development that Controls Real Life Devices and AR/VR Devices by Thinking Only, Project Identification No: 1711193852) and the Ministry of Science and Technology (MSIT) Integrated Medical Device Research and Development Project (Project Name: Development of Digital Motion Model of Upper Limb-Brain-Nerve-Musculoskeletal System and Core Technology for Diagnosis of Smart Brain Lesion Disease Utilizing XR and Wearable (SMART-LIMB), Project Identification Number: 1711196704).

### Description of the Related Art

Rehabilitation training refers to a program provided to restore a physical function in case that a motion function of a body part has been reduced because of an injury. In general, the rehabilitation training focuses on improving muscular strength and restoring muscular nerves by repeatedly moving the dysfunctional body part under the supervision of a medical practitioner or a rehabilitation trainer.

Recently, technologies have been developed to assist in rehabilitation training by means of computer devices and programs. For example, when the computer device, instead of the medical practitioner, provides instructions to a patient through a display, the patient performs a body action on the basis of the corresponding instruction, and a sensor detects the body action to determine whether the patient has performed the correct action. The computer may provide feedback to the patient or the medical practitioner to assist in the patient's rehabilitation training.

In particular, in upper limb motion control, neural commands for a central nervous system are very important because the neural commands determine how many muscles of the upper limb are used in a harmonious pattern through synergy (motor program). As the number of stroke patients is rapidly increasing because of the aging society, medical expenses are continuously increasing, and the socioeconomic costs due to the stroke and the burden of family support have reached KRW 4.8 trillion. Impaired motion control of the upper limb, which is essential for performing tasks related to daily life, caused by stroke or brain lesion disorder, is highly affected by abnormal muscle synergy and spasticity.

The initial studies have been conducted at the Shirley Ryan Ability Lab. (formerly known as the Rehabilitation Institute of Chicago (RIC)) in the U.S. to investigate abnormal synergy in stroke patients from the point of view of a motor program. However, there is a lack of research on how to use abnormal synergy for diagnosis. In addition, the upper limb evaluation is mainly performed with ordinal scales such as Fugl-Myer score and Modified Ashworth Scale, which leaves room for rater intervention and makes it impossible to distinguish between scores. For this reason, there was a lack of a precise, accurate, objective, and numerical upper limb motion control ability evaluation method. In addition, various types of rehabilitation robots have been developed, but most of the rehabilitation robots are aimed only at providing rehabilitation training. That is, the upper limb motion control technology in the related art is not related to digital evaluation methods and devices that are based on a deep understanding of the upper limb motion control ability as a whole. There is a lack of a method and apparatus for configuring a digital motion model for estimating abnormal muscle synergy.

In order to solve the above-mentioned problems, technologies have been proposed to estimate various types of abnormal muscle synergy. With reference to Korean Patent Laid-Open No. 10-2020-0104025, the subject performs a particular task through a rectilinear motion and identifies a plurality of muscle synergies related to the particular task on the basis of a plurality of electromyograms measured by the biosignal measurement device. Further, the motion ability of the subject is measured on the basis of an upper limb joint angle of the subject, which is extracted from the task, and the intensity of a force, and the muscle synergy at any time point, at which the motion ability deteriorates, is extracted as abnormal muscle synergy. However, this technology is limited only to a particular mechanism because a forward rectilinear motion may be performed inside the body, it is impossible to perform various daily activities, such as drawing circles, in addition to the rectilinear motion, and a synergy result may highly vary depending on the task. For this reason, there is still a problem in that it is difficult to perform various operations required for daily life.

### Document of Related Art

(Patent Document 0001) Korean Patent Laid-Open No. 10-2020-0104025 (Sep. 3, 2020)

### SUMMARY OF THE INVENTION

Accordingly, the present application has been made in an effort to solve the above-mentioned problems, and an object is to provide visual cues and feedback capable of creating repetitive tasks by implementing visual feedback used with XR without the need to wear a robotic device.

It is possible to perform various daily activities, such as drawing circles, in addition to rectilinear motions without being limited to a particular mechanism.

It is possible to recognize the user's weakness through a personal digital motion model through the extraction of abnormal muscle synergy and to recognize a disability degree through upper limb disability element evaluation.

An embodiment of the present application provides an apparatus for evaluating XR-based upper limb motion control and motion control ability, the apparatus including: a head mount display (HMD) configured to be worn on a user's head part and including a display for providing visual feedback information capable of monitoring a visual cue and a repetitive motion of muscle synergy or a direction and magnitude of a force that are contents for XR (eXtended Reality)-based multi-joint rehabilitation training of an upper limb; a measurement part configured to measure activation of the user's muscle or a direction and magnitude of a force applied by the user and provide tactile biofeedback; a synergy evaluation part configured to evaluate the user's muscle synergy on the basis of the measured motion of a joint angle or the measured direction and magnitude of the force; a visual cue control part configured to control the visual cue on the basis of the evaluated muscle synergy; and a composite biofeedback part configured to control the visual feedback and tactile biofeedback on the basis of the evaluated muscle synergy.

In the embodiment, the multi-joint rehabilitation training of the upper limb may be a repetitive activity related to the user's daily life.

In the embodiment, the apparatus may further include: a disability degree recognition part configured to extract abnormal muscle synergy by comparing the evaluated muscle synergy and a normal muscle synergy indicator pre-stored in a database and recognize a degree of the user's upper limb disability.

In the embodiment, the apparatus may further include: a training task generation part configured to create a training task suitable for the user's upper limb rehabilitation training on the basis of a recognized disability degree.

In the embodiment, the measurement part may be configured to receive information related to the direction and magnitude of the force from a force measurement tool configured such that the user grips the force measurement tool with hand and apply a force.

In the embodiment, the synergy evaluation part may evaluate, in real time, the muscle synergy by analyzing the number of user's muscle synergies, a synergy weight in a spatial dimension, and an activity coefficient in a temporal dimension.

In the embodiment, the synergy evaluation part may analyze the muscle synergy at a cortical level, a spinal level, and a muscular level.

In the embodiment, the visual feedback information may include diagnosis information related to a part with a weakened motion control ability based on the user's abnormal muscle synergy or information related to an abnormal muscle synergy mitigation degree and a disability mitigation degree according to a process of a rehabilitation motion.

In the embodiment, the tactile biofeedback may be based on a vibrator that induces the user to perform a repetitive task.

In the embodiment, the apparatus may further include: a fixing part configured to fix a part of the user's body so that a force, which is applied by a body part, except for a muscle synergy evaluation part, does not intervene.

Another embodiment of the present application provides a method of evaluating XR-based upper limb motion control and motion control ability performed by a processor, the method including: providing and displaying, by a head mount display (HMD) configured to be worn on a user's head part, visual feedback information capable of monitoring a visual cue and a repetitive motion of muscle synergy or a direction and magnitude of a force that are contents for XR (eXtended Reality)-based multi-joint rehabilitation training of an upper limb; measuring activation of the user's muscle or a direction and magnitude of a force applied by the user; evaluating the user's muscle synergy on the basis of the measured muscle activation or the measured direction and magnitude of the force; controlling the visual cue on the basis of the evaluated muscle synergy; and controlling the visual feedback and tactile biofeedback on the basis of the evaluated muscle synergy.

Still another embodiment of the present application provides a computer program stored in a computer-readable recording medium and configured to perform the method of evaluating XR-based upper limb motion control and motion control ability.

According to the apparatus and method for evaluating XR-based upper limb motion control and motion control ability according to the embodiment of the present application, it is possible to develop personalized upper limb motion disability evaluation/diagnosis assistance and rehabilitation training programs by inducing the patient's degree of immersion by using the XR and wearable devices.

It is possible to evaluate the motion control ability required for the user to perform the activity related to daily life by using the upper limbs (hands, arms, shoulders, etc.) in various environment, such that it is possible to provide the motion ability therapy customized for the upper limb disability. It is possible to objectively diagnose the part with the weakened motion control ability in accordance with the quantitative muscle synergy evaluation method and to improve the efficiency of the rehabilitation training.

The effects of the present application are not limited to the aforementioned effects, and other effects, which are not mentioned above, will be clearly understood by those skilled in the art from the claims.

### BRIEF DESCRIPTION OF THE DRAWINGS

FIG. 1 is a configuration view illustrating some components and operations of an apparatus for evaluating XR-based upper limb motion control and motion control ability according to an embodiment.

FIG. 2 is a block diagram of the apparatus for evaluating XR-based upper limb motion control and motion control ability according to the embodiment.

FIG. 3 is a flowchart illustrating steps of a method of evaluating XR-based upper limb motion control and motion control ability according to the embodiment.

FIGS. 4A to 4C are views illustrating a method of evaluating muscle synergy by a synergy evaluation part according to the embodiment.

FIG. 5 is a view illustrating an example for explaining a concept of muscle synergy within roles and configurations of a nervous system and a musculoskeletal system that adjust the upper limb motion control and the motion control ability according to the embodiment.

FIG. 6 is a view illustrating an example for providing visual feedback on the basis of the evaluated muscle synergy according to the embodiment.

FIGS. 7A and 7B are views illustrating examples for providing visual cues and visual feedback by using an HMD according to the embodiment.

### DETAILED DESCRIPTION OF THE INVENTION

The terms used in the present specification are selected from general terms currently widely used in the art in consideration of functions, but the terms may vary according to the intention of those skilled in the art, practice, or new technology in the art. Further, specified terms are selected arbitrarily by the applicant, and in this case, the detailed meaning thereof will be described in the description of the present specification. Thus, the terms used in the present specification should be defined based not on simple names but on the substantial meaning of the terms and the overall description of the present specification.

In addition, the embodiments disclosed in the present specification may have a configuration that is hardware as a whole, hardware partially, software partially, or software as a whole. In the present specification, the term “unit,” “device,” “or “system” may refer to computer-related entity such as hardware, software, or a combination of hardware and software. For example, the term “unit,” “device,” or “system” may refer to hardware, which constitutes a part or the entirety of a platform, and/or software such as an application (application) for operating the hardware.

Hereinafter, embodiments will be described in detail with respect to the accompanying drawings and the contents illustrated in the accompanying drawings, but the scope intended to be claimed is not restricted or limited to the embodiments.

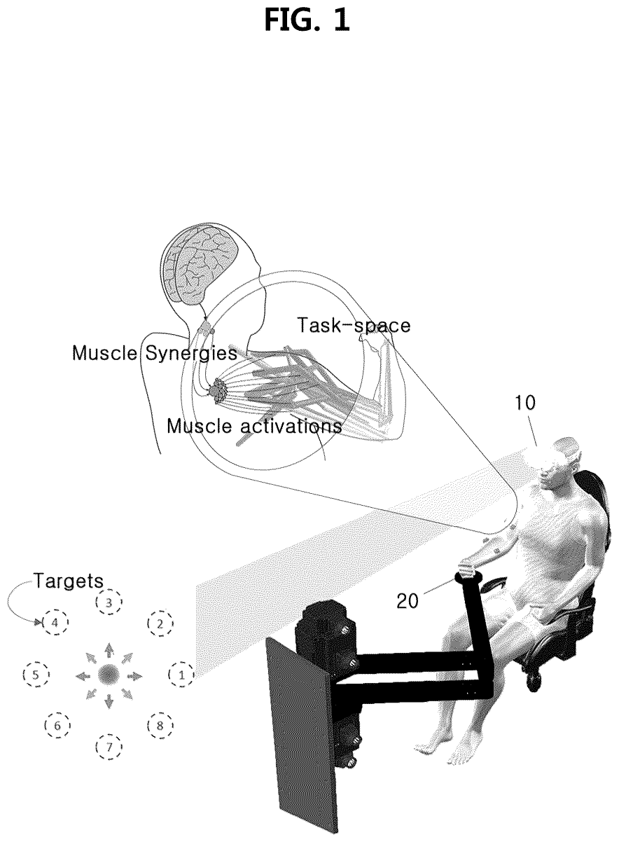

FIG. 1 is a configuration view illustrating some components and operations of an apparatus for evaluating XR-based upper limb motion control and motion control ability according to an embodiment.

With reference to FIG. 1, the apparatus includes an HMD 10 configured to be worn on a user's head part to evaluate the XR-based upper limb motion control and the motion control ability, and a measurement part 20. The HMD 10 refers to a display device capable of allowing the user to view virtual objects and reality together. The HMD 10 provides visual cues, which are contents for multi-joint rehabilitation training of upper limbs, in a manner of XR (eXtended Reality) including virtual reality (VR), mixed reality (MR), augmented reality (AR), and the like. The XR includes an environment in which virtuality and reality are tightly connected, i.e., an environment in which the user repeatedly performs activities in daily life. The visual cues show the user a target that is a target force direction. The visual cues may allow the user to perform various operations including drawing circles in a clockwise or counterclockwise direction, leftward/rightward motion adjustment, velocity adjustment, magnitude adjustment, rectilinear motions, and the like. In this case, the direction, the velocity, the magnitude of the activity, and the like may be selected. In the present specification, the user is a person who is intended to be subjected to the evaluation of motion control ability through the evaluation of muscle synergy of the body part by the apparatus. For example, the user may be a patient who has suffered from an injury, has deterioration in function of a part of the body, and needs rehabilitation training or an athlete who wants a quantitative evaluation of their physical performance. The target force direction means a direction that is a criterion for evaluating the user's muscle synergy, i.e., a particular direction in which the user applies a force by using the body part in response to the instruction.

In addition, the HMD 10 provides visual feedback information capable of monitoring, in real time, a direction and magnitude of a repetitive motion or force of the muscle synergy. The HMD 10 is formed in a shape that surrounds the eyeballs and the front side of the head part. Depending on the environment, the HMD 10 induces the user's immersion by providing the user with contents including VR for 100% virtual reality, MR including some reality information, and AR for manipulation based on the real environment and providing visual feedback.

In another embodiment, the HMD 10 may also be configured to output a voice, which indicates a target force direction, instead of an image by using a voice output device (not illustrated) such as a speaker. For example, the speaker may provide the target force direction to the user by outputting an instruction voice such as “Apply Force Downward”.

The measurement part 20 is a component configured to measure a movement of the user's joint angle or a direction and magnitude of a force applied by the user and provide tactile biofeedback. With reference to FIG. 1, when the user applies a predetermined amount of force to a force measurement tool in a force direction of a target displayed on the display of the HMD 10, the measurement part 20 measures, in real time, the movement of a joint angle applied to the force measurement tool by the user and the direction and magnitude of the force and stores measurement data in a storage device such as a memory. The measurement part 20 may generate muscle activation by transmitting signals through neural circuits at a cortical level and measure the signals applied to the muscles by means of electromyograms.

According to the embodiment, the measurement part 20 may use the force measurement tool, which is gripped by the user's hand to apply a force to the force measurement tool, so that the synergy evaluation part evaluates the muscle synergy on the user's upper body part (more specifically, hands, arms, and shoulders). As illustrated in FIG. 1, the measurement part 20 according to the embodiment may be manufactured in a shape that allows the user to grip the measurement part 20 with his/her hand and move the measurement part 20 in various directions (e.g., a rod shape vertically standing on a floor). The tool may be equipped with a sensor (e.g., a six-axis sensor or the like) capable of detecting a change in an inclination direction of a rod, a velocity, torque, or the like. The information detected by the sensor, i.e., the information related to the direction and magnitude of the force is transmitted, in the form of an electrical signal, to the synergy evaluation part. The measurement part 20 measures the direction and magnitude of the force currently applied to the measurement tool on the basis of an input signal.

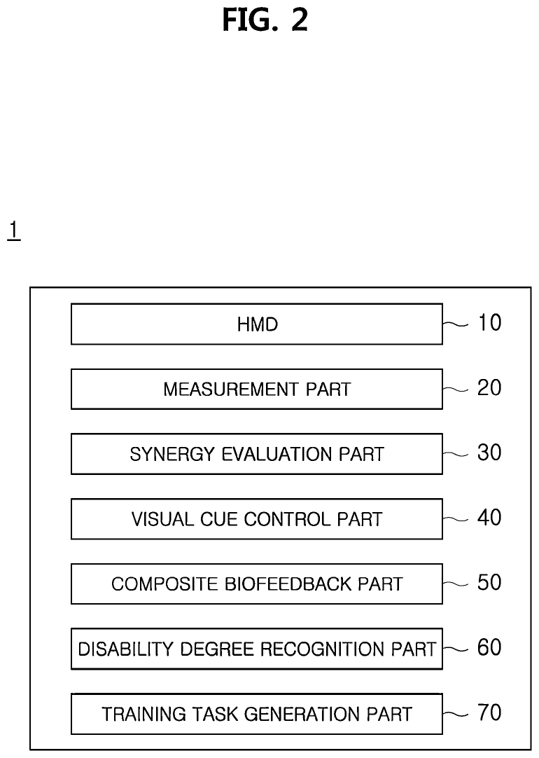

FIG. 2 is a block diagram of the apparatus for evaluating XR-based upper limb motion control and motion control ability according to the embodiment.

With reference to FIG. 2, the apparatus 1 for evaluating XR-based upper limb motion control and motion control ability (hereinafter, referred to as an “upper limb motion control apparatus”) may include the HMD 10, the measurement part 20, a synergy evaluation part 30, a visual cue control part 40, a composite biofeedback part 50, a disability degree recognition part 60, and a training task generation part 70.

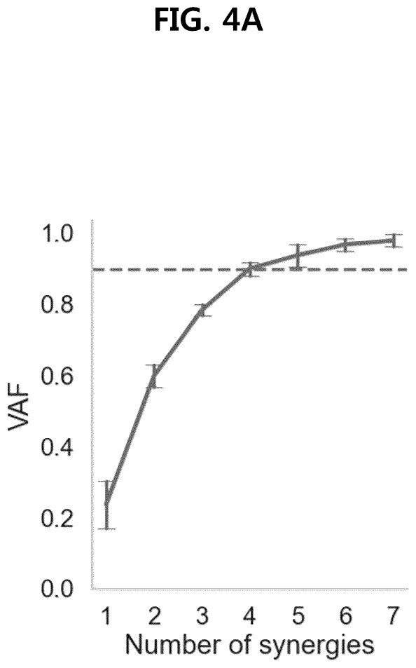

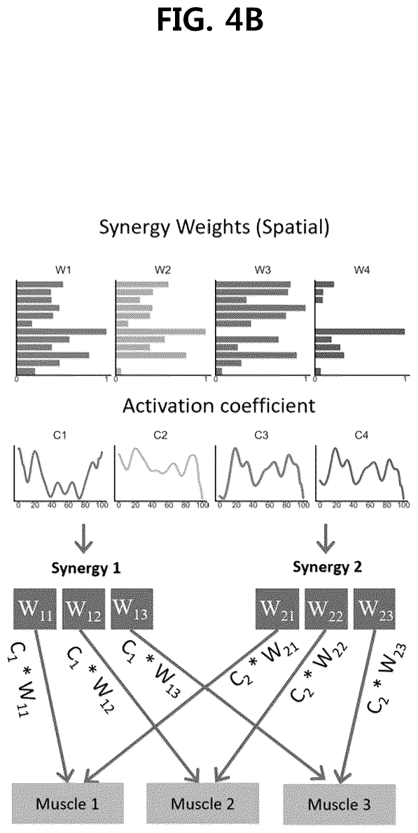

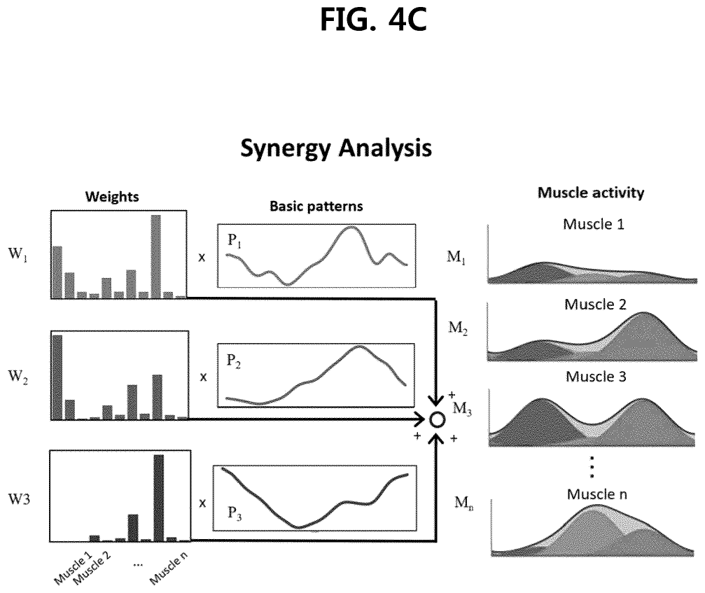

The synergy evaluation part 30 evaluates the user's muscle synergy on the basis of the electromyogram measured by the measurement part 20, the movement of the joint angle, or the direction and magnitude of the force. A specific method of evaluating the muscle synergy by the synergy evaluation part 30 will be described in detail with reference to FIGS. 4A to 4C.

The visual cue control part 40 controls the visual cue on the basis of the evaluated muscle synergy. The visual cue may show the user the target that is the target force direction. The visual cues may allow the user to perform various operations including drawing circles, rectilinear motions, and the like. The target force direction means a direction that is a criterion for evaluating the user's muscle synergy, i.e., a particular direction in which the user applies a force by using the body part in response to the instruction.

In the embodiment, for example, the visual cue control part 40 may perform control to sequentially output images, which indicate the target force direction, on a display in response to an evaluation program through the HMD 10 or the display device such as the display. On the basis of the result of evaluating the user's muscle synergy, the visual cue control part 40 may use images having various shapes for further improving the upper limb motion by allowing the user to perform activities in particular directions on a weak part or a part from which the abnormal muscle synergy is extracted.

The composite biofeedback part 50 controls the visual feedback information and the tactile biofeedback on the basis of the evaluated muscle synergy. The visual feedback information includes diagnosis information related to the part with the weakened motion control ability based on the user's abnormal muscle synergy or information related to an abnormal muscle synergy mitigation degree and a disability mitigation degree according to the process of the rehabilitation motion. The diagnosis information related to the weak part may be created on the basis of a database that stores data related to an injury to the body part and the degradation of the physical function. For example, it may be difficult to perform an activity of pushing an object forward with the right hand in case that a rotator cuff of the right shoulder is injured. When the result of evaluating the muscle synergy indicates that the abnormal muscle synergy is extracted when the user performs the activity of pushing an object forward with the right hand, diagnosis information, which indicates that the rotator cuff is weak, may be provided to the user.

The tactile biofeedback may be based on a vibrator that induces the user to perform repetitive tasks. For example, in case that the user needs to perform the activity of drawing circles a total of ten times, the vibration is generated until the user performs the activity ten times, such that the instruction may be made so that the user continuously performs the activity.

The disability degree recognition part 60 extracts the abnormal muscle synergy by comparing the evaluated muscle synergy and a normal muscle synergy indicator pre-stored in the database and recognizes a degree of the user's upper limb disability. For example, when the user performs the activity of drawing circles, the muscle synergy evaluated by the synergy evaluation part 30 is compared with the normal muscle synergy indicator related to the activity of drawing circles pre-stored in the database. It can be said that the disability degree is large when the difference is large.

The training task generation part 70 creates a training task suitable for the user's upper limb rehabilitation training on the basis of the recognized disability degree. For example, when the disability degree related to the activity of drawing circles is large, it is possible to create a training task capable of reinforcing muscles required to perform the activity of drawing circles.

According to the additional embodiment of the present application, the upper limb motion control apparatus 1 may further include a fixing part configured to fix a part of the user's body so that a force, which is applied by the body part, except for the muscle synergy evaluation part, does not intervene. This is because the ability to apply the force may vary depending on the posture of the human body. For example, in order to evaluate the muscle synergy of the right arm, the chest part may be fixed by the fixing part so that the muscles, e.g., the muscles of the right chest, except for the muscles of the right arm, do not intervene. For example, like a strap, the fixing part may be configured to be easily attachable to the user's body or clothes.

The HMD 10, the measurement part 20, the synergy evaluation part 30, the visual cue control part 40, the composite biofeedback part 50, the disability degree recognition part 60, the training task generation part 70, and the additional components are distinguished and described to make it easy to understand the function and role of the upper limb motion control apparatus 1. The above-mentioned components need not be necessarily and independently implemented by a separate device or program. That is, all the components may be implemented by a single processor provided in a single computer, or independently implemented by a plurality of computers or a plurality of processors. In addition, the HMD 10 may be interpreted as a comprehensive meaning including elements for connecting the display device, such as the display, to the computer. Likewise, the measurement part 20 may also be interpreted as a comprehensive meaning including elements for connecting the measurement tool to the computer.

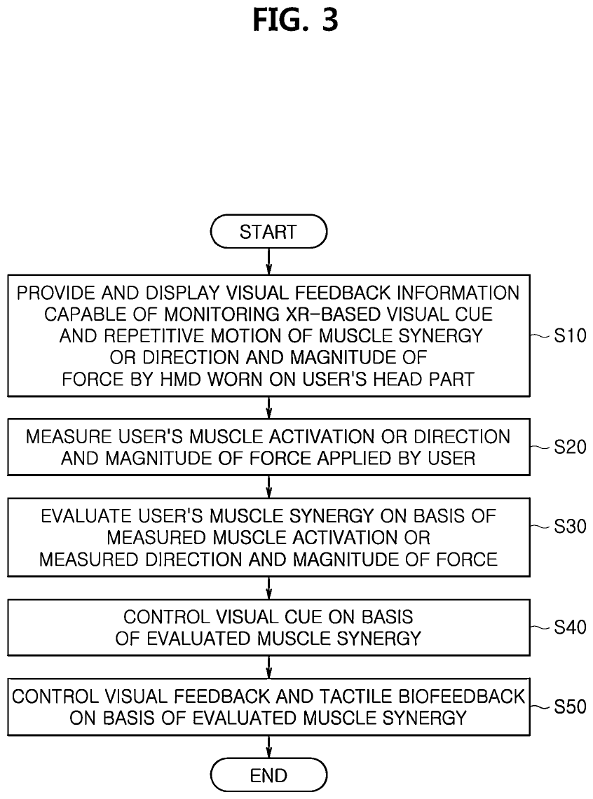

FIG. 3 is a flowchart illustrating steps of a method of evaluating XR-based upper limb motion control and motion control ability according to the embodiment. The method of evaluating the XR-based upper limb motion control and the motion control ability (hereinafter, referred to as an “upper limb motion control method”) may be performed by the processor provided in the computer. Respective steps may be implemented by a single processor provided in a single computer, or independently implemented by a plurality of computers or a plurality of processors.

With reference to FIG. 3, first, a step of providing and displaying visual feedback information capable of monitoring the visual cues and the repetitive motion of the muscle synergy or the direction and magnitude of the force, which are contents for the multi-joint rehabilitation training of the XR-based upper limb, is performed by the HMD 10 worn on the user's head part (S10). As illustrated in FIG. 1, the visual feedback information capable of monitoring, in real time, the visual cues and the repetitive motion of the muscle synergy or the direction and magnitude of the force, which are the contents for the multi-joint rehabilitation training of the upper limb in the XR manner, is provided on the display of the HMD 10.

Next, a step of measuring the motion of the user's joint angle or the direction and magnitude of the force applied by the user is performed (e.g., by the measurement part 20) (S20). As illustrated in FIG. 1, the measurement part 20 according to the embodiment may be manufactured as the force measurement tool in a shape that allows the user to grip the measurement part 20 with his/her hand and move the measurement part 20 in various directions. The tool may be equipped with a sensor capable of detecting a change in an inclination direction of a rod, a velocity, torque, or the like. The information detected by the sensor, i.e., the information related to the direction and magnitude of the force is converted into an electrical signal, and the electrical signal is transmitted to the processor. The processor measures the angle of the joint and the direction and magnitude of the force currently applied to the measurement tool on the basis of the input signal.

Next, a step of evaluating the user's muscle synergy on the basis of the measured muscle activation or the measured direction and magnitude of the force is performed (e.g., by the synergy evaluation part 30) (S30). As described above with reference to FIG. 2, the specific method of step S30 of evaluating the muscle synergy will be described in detail with reference to FIGS. 4A to 4C.

Next, a step of controlling the visual cue on the basis of the evaluated muscle synergy is performed (e.g., by the visual cue control part 40) (S40). In step S40 of controlling the visual cue, the visual cue is controlled on the basis of the evaluated muscle synergy. The visual cue may show the user the target that is the target force direction. The visual cues may allow the user to perform various operations including drawing circles, rectilinear motions, and the like.

Next, a step of controlling the visual feedback and the tactile biofeedback on the basis of the evaluated muscle synergy is performed (e.g., by the composite biofeedback part 50) (S50). The visual feedback information includes diagnosis information related to the part with the weakened motion control ability based on the user's abnormal muscle synergy or information related to an abnormal muscle synergy mitigation degree and a disability mitigation degree according to the process of the rehabilitation motion. The diagnosis information related to the weak part may be created on the basis of a database that stores data related to an injury to the body part and the degradation of the physical function. The tactile biofeedback may be based on a vibrator that induces the user to perform repetitive tasks.

FIGS. 4A to 4C are views illustrating a method of evaluating muscle synergy by the synergy evaluation part according to the embodiment.

With reference to FIGS. 4A to 4C, the synergy evaluation part 30 may evaluate, in real time, the muscle synergy by analyzing the number of user's muscle synergies, a synergy weight in a spatial dimension, and an activity coefficient in a temporal dimension. For example, during the user's particular activity, the synergy evaluation part 30 may extract the number of muscles indicating the muscle synergy and extract the activity coefficient indicating the activity degree of the muscle synergy for each time zone. The muscle synergy may be expressed as a weight for each muscle depending on the combination of the muscles used to perform the particular activity. The weight may be expressed as a value of 0 to 1 depending on a degree to which a particular muscle is used a large number of times in a spatial dimension.

The synergy evaluation part 30 may analyze the muscle synergy at a cortical level, a spinal level, and a muscular level. The cortical level is the highest level of neuromuscular control. The cortical level initiates and adjusts complex and numerical motions. The important three zones of the cortex include a primary motion cortex, a pre-motion area, and an auxiliary motion area. The primary motion cortex receives proprioceptive information, the pre-motion area organizes and the pre-motion, and the auxiliary motion area programs muscle groups for complex movements.

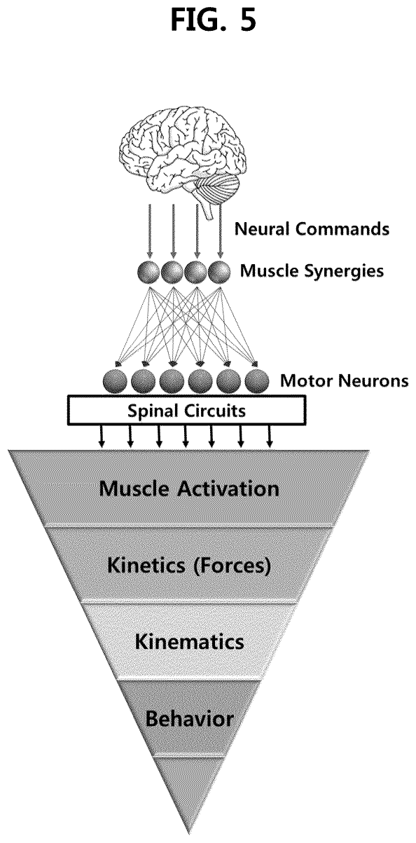

FIG. 5 is a view illustrating an example for explaining a concept of muscle synergy within roles and configurations of a nervous system and a musculoskeletal system that adjust the upper limb motion control and the motion control ability according to the embodiment.

With reference to FIG. 5, the measurement part generates muscle activation by transmitting signals through neural circuits at the cortical level and measures the signals applied to the muscles by means of electromyograms. On the basis of the measurement, the synergy evaluation part may evaluate the motor program, i.e., the muscle synergy shown at the cortical level and the spinal level.

The adjustment at the spinal level is related to the isolated spinal cord reflexes and directly affected by centripetal information from joint receptors. This reflex is very fast, involuntary, and unconscious. The reflex occurs in coordination between the agonistic and antagonist muscles. When the agonistic muscle contracts, the antagonist automatically relaxes. An example of this flex includes the extensor reflex, i.e., a sudden motion of the knee that occurs when the knee cap tendon is lightly tapped. That is, the synergy evaluation part 30 may also evaluate the user's proprioception by analyzing the muscle synergy at the cortical level, the spinal level, and the muscular level.

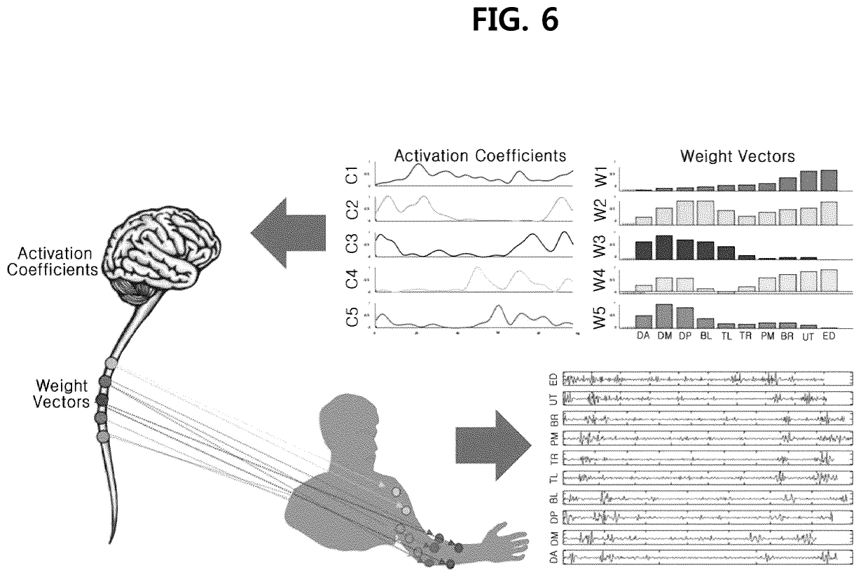

FIG. 6 is a view illustrating an example for providing visual feedback on the basis of the evaluated muscle synergy according to the embodiment.

With reference to FIG. 6, the HMD 10 provides visual feedback information capable of monitoring, in real time, a direction and magnitude of a repetitive motion or force of the muscle synergy. The user's immersion is induced by providing the user with the content configured to be based on XR and providing the visual feedback. For example, the visual feedback visualizes the number of muscle synergies, the weight vector for each time, and the activity coefficient on the display of the HMD 10 by analyzing, in real time, the muscle synergy of the synergy evaluation part 30 and visualizes the activity for each muscle, thereby enabling the creation of the upper limb segment for increasing the immersion.

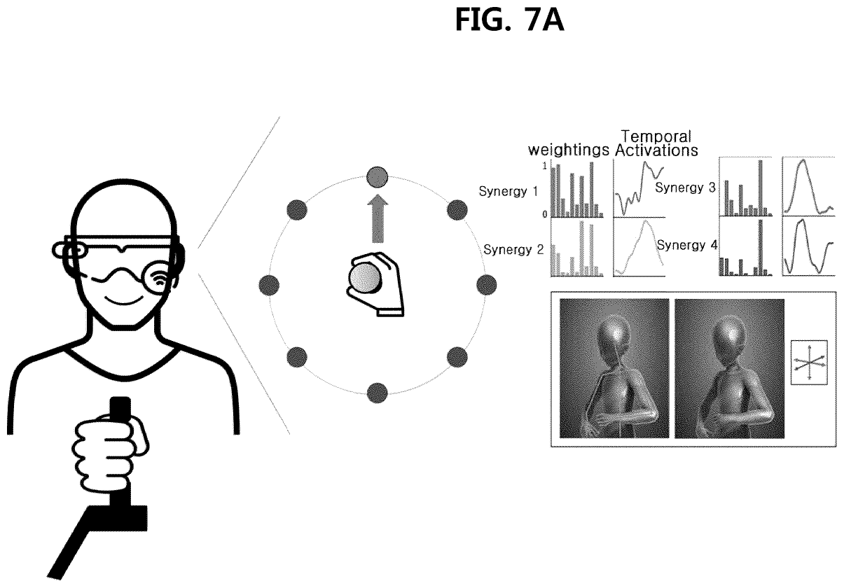

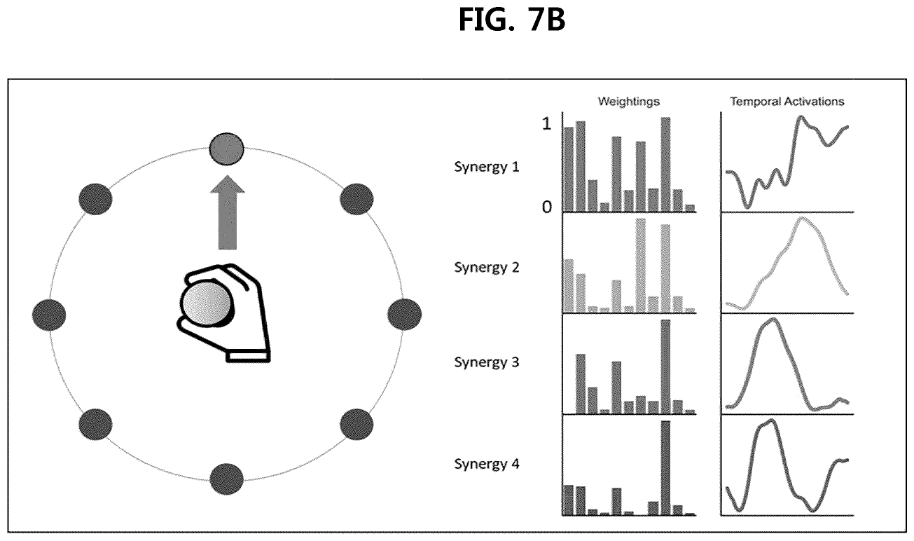

FIGS. 7A and 7B are views illustrating examples for providing visual cues and visual feedback by using an HMD according to the embodiment.

With reference to FIGS. 7A and 7B, the HMD 10 provides the visual cue, which is the content for the multi-joint rehabilitation training of the upper limb, in the XR manner. The visual cue allows the user to perform the multi-joint rehabilitation training activity of the upper limb, i.e., the repetitive activity related to the user's daily life. The visual cue may show the user the target that is the target force direction. The visual cues may allow the user to perform various operations including drawing circles, rectilinear motions, and the like. In addition to the visual cue, the HMD 10 may provide visual feedback information capable of monitoring, in real time, the direction and magnitude of the repetitive motion or force of the muscle synergy. The visual feedback information may include diagnosis information related to the part with the weakened motion control ability based on the user's abnormal muscle synergy or information related to an abnormal muscle synergy mitigation degree and a disability mitigation degree according to the process of the rehabilitation motion.

According to the above-mentioned upper limb motion control apparatus and method, it is possible to develop personalized upper limb motion disability evaluation/diagnosis assistance and rehabilitation training programs by inducing the patient's degree of immersion by using the XR and wearable devices. It is possible to evaluate the motion control ability required for the user to perform the activity related to daily life by using the upper limbs (hands, arms, shoulders, etc.) in various environment, such that it is possible to provide the motion ability therapy customized for the upper limb disability. It is possible to objectively diagnose the part with the weakened force control ability in accordance with the quantitative muscle synergy evaluation method and to improve the efficiency of the rehabilitation training.

The upper limb motion control method according to the embodiment may be implemented as applications or may be in the form of program instructions executable through various computer elements, and then recorded in a computer-readable recording medium. The computer-readable recording medium may include program instructions, data files, data structures, or the like, in a stand-alone form or in a combination thereof.

Examples of the computer-readable recording medium may include magnetic media, such as a hard disk, a floppy disk and a magnetic tape, optical recording media, such as CD-ROM and DVD, magneto-optical media, such as a floptical disk, and hardware devices, such as ROM, RAM and flash memory, which are specifically configured to store and run program instructions.

While the present application has been described above with reference to the embodiments, it may be understood by those skilled in the art that the present application may be variously modified and changed without departing from the spirit and scope of the present application disclosed in the claims.

What is claimed is:

1. An apparatus for evaluating XR-based upper limb motion control and motion control ability, the apparatus comprising:
a head mount display (HMD) configured to be worn on a user's head part and including a display for providing visual feedback information capable of monitoring a visual cue and a repetitive motion of muscle synergy or a direction and magnitude of a force that are contents for XR (eXtended Reality)-based multi-joint rehabilitation training of an upper limb;
a measurement part configured to measure activation of the user's muscle or a direction and magnitude of a force applied by the user and provide tactile biofeedback;
a synergy evaluation part configured to evaluate the user's muscle synergy on the basis of the measured motion of a joint angle or the measured direction and magnitude of the force;
a visual cue control part configured to control the visual cue on the basis of the evaluated muscle synergy; and
a composite biofeedback part configured to control the visual feedback and tactile biofeedback on the basis of the evaluated muscle synergy.

2. The apparatus of claim 1, wherein the multi-joint rehabilitation training of the upper limb is a repetitive activity related to the user's daily life.

3. The apparatus of claim 1, further comprising:
a disability degree recognition part configured to extract abnormal muscle synergy by comparing the evaluated muscle synergy and a normal muscle synergy indicator pre-stored in a database and recognize a degree of the user's upper limb disability.

4. The apparatus of claim 3, further comprising:
a training task generation part configured to create a training task suitable for the user's upper limb rehabilitation training on the basis of a recognized disability degree.

5. The apparatus of claim 1, wherein the measurement part is configured to receive information related to the direction and magnitude of the force from a force measurement tool configured such that the user grips the force measurement tool with hand and apply a force.

6. The apparatus of claim 1, wherein the synergy evaluation part evaluates, in real time, the muscle synergy by analyzing the number of user's muscle synergies, a synergy weight in a spatial dimension, and an activity coefficient in a temporal dimension.

7. The apparatus of claim 1, wherein the synergy evaluation part analyzes the muscle synergy at a cortical level, a spinal level, and a muscular level.

8. The apparatus of claim 1, wherein the visual feedback information includes diagnosis information related to a part with a weakened motion control ability based on the user's abnormal muscle synergy or information related to an abnormal muscle synergy mitigation degree and a disability mitigation degree according to a process of a rehabilitation motion.

9. The apparatus of claim 1, wherein the tactile biofeedback is based on a vibrator that induces the user to perform a repetitive task.

10. The apparatus of claim 1, further comprising:
a fixing part configured to fix a part of the user's body so that a force, which is applied by a body part, except for a muscle synergy evaluation part, does not intervene.

11. A method of evaluating XR-based upper limb motion control and motion control ability performed by a processor, the method comprising:
providing and displaying, by a head mount display (HMD) configured to be worn on a user's head part, visual feedback information capable of monitoring a visual cue and a repetitive motion of muscle synergy or a direction and magnitude of a force that are contents for XR (eXtended Reality)-based multi-joint rehabilitation training of an upper limb;
measuring activation of the user's muscle or a direction and magnitude of a force applied by the user;
evaluating the user's muscle synergy on the basis of the measured muscle activation or the measured direction and magnitude of the force;
controlling the visual cue on the basis of the evaluated muscle synergy; and
controlling the visual feedback and tactile biofeedback on the basis of the evaluated muscle synergy.

12. A computer program stored in a computer-readable recording medium and configured to perform the method according to claim 11 of evaluating XR-based upper limb motion control and motion control ability.
